(19) United States
(12) Patent Application Publication
(10) Pub. No.: US 2018/0042498 A1
(43) Pub. Date: Feb. 15, 2018

(54) PHOTOELECTRIC PULSE WAVE SENSOR AND DETECTION APPARATUS

(71) Applicant: BOE TECHNOLOGY GROUP CO., LTD., Beijing (CN)

(72) Inventor: Ruisi Chen, Beijing (CN)

(21) Appl. No.: 15/534,944

(22) PCT Filed: May 31, 2016
(86) PCT No.: PCT/CN2016/084034

§ 371 Date: Jun. 9, 2017

(30) Foreign Application Priority Data
Mar. 10, 2016 (CN) 201610137228.4

(51) Int. Cl. A61B 5/021; A61B 5/026; A61B 5/00; A61B 5/024

(52) U.S. Cl. CPC A61B 5/02108; A61B 5/02444; A61B 5/0261; A61B 5/7203; A61B 5/02438

(57) ABSTRACT

The present disclosure provides a photoelectric pulse wave sensor including a substrate, a protrusion structure, a transmission post, a measurement light source and a photoelectric detector, wherein, the protrusion structure is protrudedly disposed on the substrate and has a through hole therein that is perpendicular to the substrate; the measurement light source and the photoelectric detector are provided side-by-side on a face of the substrate that is right below the through hole, a shape and a size of the transmission post are matched to a shape and a size of the through hole and the transmission post is fixed in the through hole. The disclosure further provides a detection apparatus including the above photoelectric pulse wave sensor.

### CROSS-REFERENCE TO RELATED APPLICATIONS

The present application claims priority to Chinese Patent Application No. 201610137228.4, filed on Mar. 10, 2016, entitled “PHOTOELECTRIC PULSE WAVE SENSOR AND DETECTION APPARATUS”, which is incorporated herein by reference in its entirety.

### BACKGROUND

### 1. Technical Field

The present disclosure relates to technical field of sensor, and particularly to a photoelectric pulse wave sensor and a detection apparatus.

### 2. Description of the Related Art

Systole and diastole of human body's heart chamber cause systole and diastole of aorta such that blood pressure is transmitted, from the root of the aorta, along the whole arterial system in form of wave, which is called pulse wave. The pulse wave presents information on several aspects such as form, intensity, velocity, and rhythm and reflects physiological and pathologic features of human body's cardiovascular form to great extent, and thus is an important physiological factor of human body. There are a plurality of existing measurement sensors for the pulse wave, including a piezoelectric sensor and a photoelectric sensor, etc. The photoelectric sensor is a pulse wave sensor based on a photoelectric volumetric method. According to Lambert-Beer law, light absorption of a substance is in proportion to its concentration at a certain wave of light. When a light with a constant wave is incident to human body's tissue, structural feature of the tissue may be reflected to a certain extent by measuring intensity of the light that has been absorbed and reflected by the human body's tissue. The photoelectric pulse wave sensor indirectly measures pulse signal by means of measurement of light transmission of the wrist or finger tip based on Lambert-Beer law. Among those, a reflective photoelectric pulse wave sensor includes a light source and a light sensitive device located on the same side thereof, and can accurately measure variation of volume within the blood vessel, and has advantages of simple structure, no damage, and good repeatability, etc. However, when measurement is performed by the photoelectric pulse wave sensor, a pressure on vascular wall causes unstable pressure variety to the light sensitive device, i.e., receiving end of a reflected light, of the sensor due to the pulse in the artery. The pressure variety will directly cause noise in the pulse wave. In addition, blood flow signal at portions such as wrist is rather weak; the vascular wall itself has flexibility that also causes unstable pressure variety at the reflected light receiving end and in turn directly leads to noise in the pulse wave. Thus, it is an existing problem to be solved urgently that how to eliminate noise caused by pressure on the vascular wall so as to increase measurement accuracy of the photoelectric pulse wave sensor.

### SUMMARY

The present disclosure provides a photoelectric pulse wave sensor comprising: a substrate, a protrusion structure, a transmission post, a measurement light source and a photoelectric detector, wherein, the protrusion structure is protrudedly disposed on the substrate and has a through hole therein that is perpendicular to the substrate; the measurement light source and the photoelectric detector are provided side-by-side on a face of the substrate that is right below the through hole, a shape and a size of the transmission post are matched to a shape and a size of the through hole and the transmission post is fixed in the through hole.

According to an exemplary embodiment of the present disclosure, the protrusion structure includes stacking layers that are composed of N layers of discs each having a central hole, the central holes of the N layers of discs being in positional correspondence with one another and forming the through hole that is perpendicular to the substrate, where N≧2. According to an exemplary embodiment of the present disclosure, 3≦N≦10.

According to an exemplary embodiment of the present disclosure, sizes of cross sections, perpendicular to a protrusion direction, of the N layers of discs decreases gradually layer by layer in a direction away from the substrate, such that the N layers of discs form a substantially tapered protrusion structure.

According to an exemplary embodiment of the present disclosure, the disc is a rectangular disc, a circular disc or an elliptical disc.

According to an exemplary embodiment of the present disclosure, the N layers of discs have a same shape, and the cross sections, perpendicular to the protrusion direction, of the N layers of discs have a same size.

According to an exemplary embodiment of the present disclosure, the disc is a rectangular disc, a circular disc or an elliptical disc, to form a pillar-shaped protrusion structure as a whole.

According to an exemplary embodiment of the present disclosure, the protrusion structure is a pillar-shaped integral structure or a substantially tapered integral structure having multiple steps.

According to an exemplary embodiment of the present disclosure, the protrusion structure is a substantially tapered integral structure and comprises N step structures (17); sizes of cross sections, perpendicular to a protrusion direction, of the N step structures decreases gradually layer by layer in a direction away from the substrate.

According to an exemplary embodiment of the present disclosure, a cross section, perpendicular to the protrusion direction, of each of the N step structures has a shape of rectangle, circle or ellipse.

According to an exemplary embodiment of the present disclosure, the through hole in the protrusion structure is located in a central position of the protrusion structure or is deviated to a side of the protrusion structure.

According to an exemplary embodiment of the present disclosure, a cross section of the through hole in the protrusion structure is in a shape of rectangle, circle or ellipse, and the transmission post is correspondingly in a shape of a cuboid, a circular cylinder or an elliptical pillar.

According to an exemplary embodiment of the present disclosure, the photoelectric pulse wave sensor further comprises: a constant-current source control circuit located in the substrate, connected to the measurement light source and configured such that an electrical current flowing through the measurement light source is kept to be constant and thus the measurement light source emits a light with stable intensity.

According to an exemplary embodiment of the present disclosure, the photoelectric pulse wave sensor further comprises: a signal modulation circuit located in the substrate, connected to the photoelectric detector and configured to filter out direct-current component of an output signal of the photoelectric detector.

According to an exemplary embodiment of the present disclosure, the protrusion structure has a greatest thickness at a position right facing the photoelectric detector, and the thickness of the protrusion structure decreases gradually as departing from the position right facing the photoelectric detector, thereby the protrusion structure presenting a substantially tapered shape.

Further, the present disclosure provides a detection apparatus comprising the abovementioned photoelectric pulse wave sensor.

### BRIEF DESCRIPTION OF THE DRAWINGS

FIG. 1 is a longitudinal cross section view of a photoelectric pulse wave sensor according to a first embodiment of the present disclosure;

FIG. 2 is a longitudinal cross section view of a photoelectric pulse wave sensor according to a second embodiment of the present disclosure;

FIG. 3 is a longitudinal cross section view of a photoelectric pulse wave sensor according to a third embodiment of the present disclosure;

FIG. 4 is a longitudinal cross section view of a photoelectric pulse wave sensor according to a fourth embodiment of the present disclosure;

FIG. 5 is a longitudinal cross section view of a photoelectric pulse wave sensor according to a fifth embodiment of the present disclosure;

FIG. 6 is a longitudinal cross section view of a photoelectric pulse wave sensor according to a sixth embodiment of the present disclosure;

FIG. 7 is a top view of a photoelectric pulse wave sensor according to a sixth embodiment of the present disclosure;

FIG. 8 is a top view of a photoelectric pulse wave sensor according to a seventh embodiment of the present disclosure;

FIG. 9 is a top view of a photoelectric pulse wave sensor according to an eighth embodiment of the present disclosure;

FIG. 10 is a top view of a photoelectric pulse wave sensor according to a ninth embodiment of the present disclosure;

FIG. 11 is a pulse wave oscillogram obtained by a photoelectric pulse wave sensor without stacking layers;

FIG. 12 is a pulse wave oscillogram obtained by the photoelectric pulse wave sensor according to the third embodiment of the present disclosure.

### DETAILED DESCRIPTION OF THE EMBODIMENTS

In order to provide a more clear understanding of objects, technique solutions and advantages of the present disclosure, the present disclosure will be further described hereinafter in detail and completely with reference to the embodiments in conjunction with the attached drawings.

Referring to FIG. 1, a longitudinal cross section view of a photoelectric pulse wave sensor according to a first embodiment of the present disclosure is shown. The photoelectric pulse wave sensor includes: a substrate 11, a pillar-shaped integral structure 20, a transmission post 13, a measurement light source 14 and a photoelectric detector (PD) 15.

In this embodiment, the pillar-shaped integral structure 20 is disposed on the substrate 11 and has a through hole in a direction perpendicular to the substrate, the measurement light source 14 and the photoelectric detector 15 are provided side-by-side on a face of the substrate that is right below the through hole, and a shape and a size of the transmission post 13 are matched to those of the through hole and the transmission post is fixed in the through hole.

As an example, the pillar-shaped integral structure has a thickness in the range from 1 mm to 10 mm, and is correspondingly in a shape of a cuboid, a circular cylinder or an elliptical pillar. The pillar-shaped integral structure is made of opacity material, such as acrylic.

As an example, the through hole in the pillar-shaped integral structure is located in a central position of the pillar-shaped integral structure, and the transmission post 13 is made of transmission material, such as glass.

As an example, the through hole may be in a shape of rectangular, circular or elliptical. Correspondingly, the transmission post 13 may be a cuboid, a circular cylinder or an elliptical pillar.

As an example, the photoelectric detector 15 may be a light sensitive resistor, a light sensitive diode, a light sensitive triode or a silicon photocell. The measurement light source 14 is a light emitting diode (LED).

As an example, the photoelectric pulse wave sensor further includes a constant-current source control circuit 18 disposed in the substrate 11, connected to the measurement light source 14 and configured such that an electrical current flowing through the measurement light source 14 is kept to be constant and thus the measurement light source 14 emits a light with stable intensity, thereby avoiding measurement error caused by fluctuation of the measurement light source 14 and further increasing measurement accuracy of pulse wave.

As an example, the photoelectric pulse wave sensor further includes a signal modulation circuit 19 disposed in the substrate 11 and connected to the photoelectric detector 15. The signal modulation circuit 19 filters out direct-current component of output signal of the photoelectric detector 15 such that the photoelectric detector 15 only contains alternating current component. In subsequent process, acquisition of pulse signal may be achieved by means of only simple amplifier circuit and low-pass filter circuit.

In the photoelectric pulse wave sensor according to the first embodiment of the present disclosure, when measuring a pulse wave, a light emitted from the measurement light source 14 is reflected by blood and the reflected light is received by the photoelectric detector 15. As the photoelectric pulse wave sensor has the pillar-shaped integral structure 20 and the pillar-shaped integral structure 20 contacts skin when it is pressed against a location including wrist or finger tip, the photoelectric pulse wave sensor applies a force to vascular wall through the pillar-shaped integral structure 20 such that pressures inside and outside the vascular wall are equal to each other and the pressure on the vascular wall is counteracted by the force applied by the pillar-shaped integral structure 20. That is, the pressure on the vascular wall will not affect the photoelectric detector 15 and thus a noise caused by the pressure on the vascular wall is eliminated, thereby increasing measurement accuracy of the pulse wave.

Referring to FIG. 2, a longitudinal cross section view of a photoelectric pulse wave sensor according to a second embodiment of the present disclosure is provided in FIG. 2. For brief, description of the same or similar technical features as those in the first embodiment is incorporated herein and will not repeatedly described.

The though hole in the pillar-shaped integral structure of the photoelectric pulse wave sensor is not located in the central position of the pillar-shaped integral structure, but is deviated to a side of the pillar-shaped integral structure.

The photoelectric pulse wave sensor according to the second embodiment may also achieve equality of the pressures inside and outside the vascular wall, and the pressure on the vascular wall is counteracted by the force applied by the pillar-shaped integral structure 20. That is, the pressure on the vascular wall will not affect the photoelectric detector 15 and the noise caused by the pressure on the vascular wall is eliminated, thereby increasing measurement accuracy on the pulse wave.

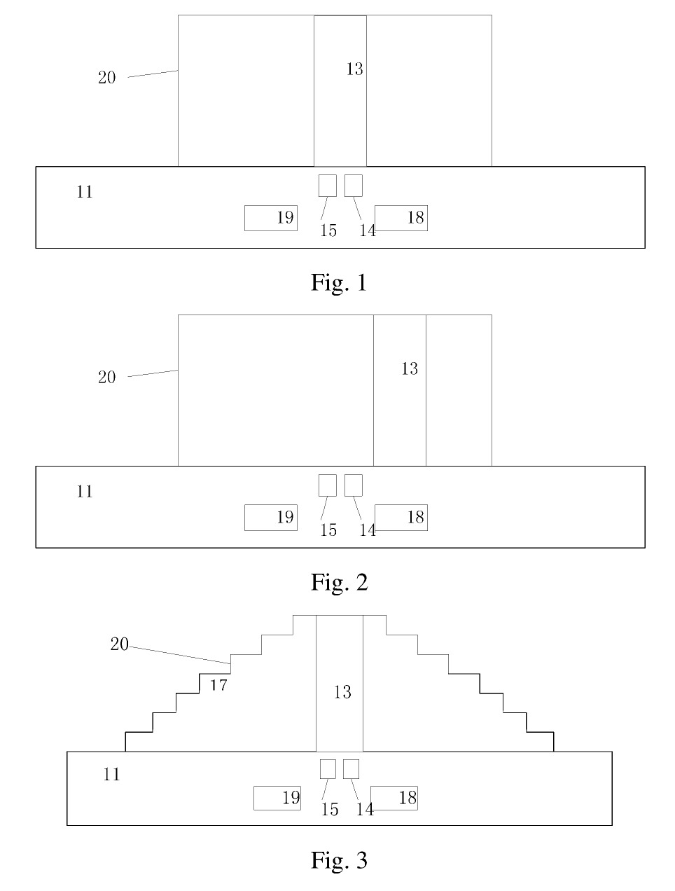

Referring to FIG. 3, a longitudinal cross section view of a photoelectric pulse wave sensor according to a third embodiment of the present disclosure is provided in FIG. 3. For brief, description of the same or similar technical features as those in any one of the above embodiments is incorporated herein and will not repeatedly described.

The integral structure 20 of the photoelectric pulse wave sensor is of substantially tapered N step structures 17, and sizes of cross sections, perpendicular to a protrusion direction, of the N step structures decreases gradually layer by layer in a direction away from the substrate.

As an example, cross sections of the N step structures have shape of rectangular, circular or elliptical, or, cross sections of the N step structures include at least two of the shapes of rectangular, circular and elliptical.

The photoelectric pulse wave sensor according to the third embodiment of the present disclosure may eliminate noise caused by the pressure on the vascular wall so as to increase measurement accuracy of the pulse wave. Further, as the photoelectric detector 15 is faced in position to a maximum pressure on the vascular wall, influence on the measurement accuracy from the pressure on the vascular wall is maximum. Further, as the vascular wall around the photoelectric detector 15 is distanced from the photoelectric detector 15, influence on the measurement accuracy is smaller. The photoelectric pulse wave sensor according to the third embodiment of the present disclosure has as a whole a substantially taper-like integral structure and a thickness of the tapered integral structure is greatest at the position right facing the photoelectric detector 15. When the tapered integral structure 20 is pressed against a skin, a force on the vascular wall applied by the tap integral structure 20 is greatest at the position right facing the photoelectric detector 15. Regarding to the vascular wall around the position right facing the photoelectric detector 15, further the vascular wall is distanced from the position right facing the photoelectric detector 15, thinner the tapered integral structure is, and thus smaller force sustained by the vascular wall is. The photoelectric pulse wave sensor according to the third embodiment of the present disclosure is provided such that external force applied on the vascular wall near the position where measurement is performed by the sensor becomes more balance, that is, the pressure on the vascular wall is counteracted more accurately by the external force applied by the tapered integral structure and thereby the measurement accuracy of the pulse wave is further increased.

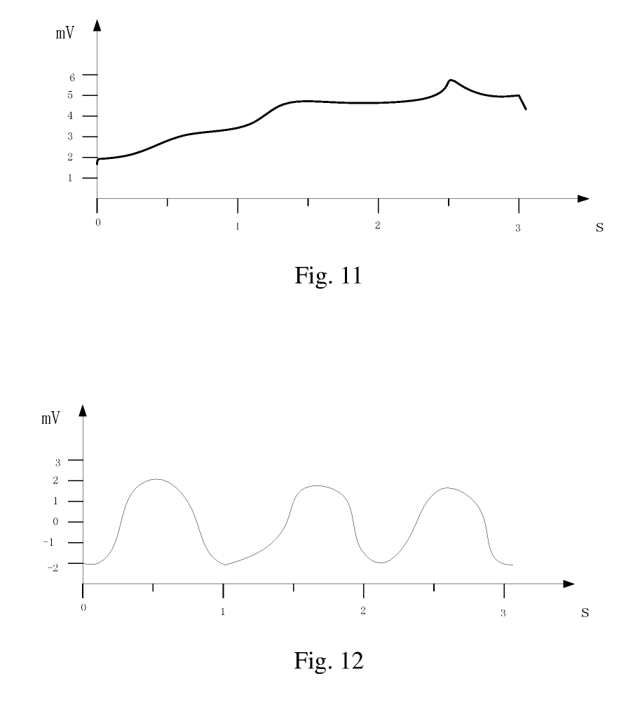

As shown in FIG. 12, a pulse wave oscillogram obtained by the photoelectric pulse wave sensor according to the third embodiment of the present disclosure is illustrated in FIG. 12. FIG. 11 shows a pulse wave oscillogram obtained without the photoelectric pulse wave sensor according to the third embodiment of the present disclosure having the tapered integral structure. It can be seen that the photoelectric pulse wave sensor according to the third embodiment of the present disclosure may eliminate noise caused by the pressure on the vascular wall so as to increase measurement accuracy of the pulse wave.

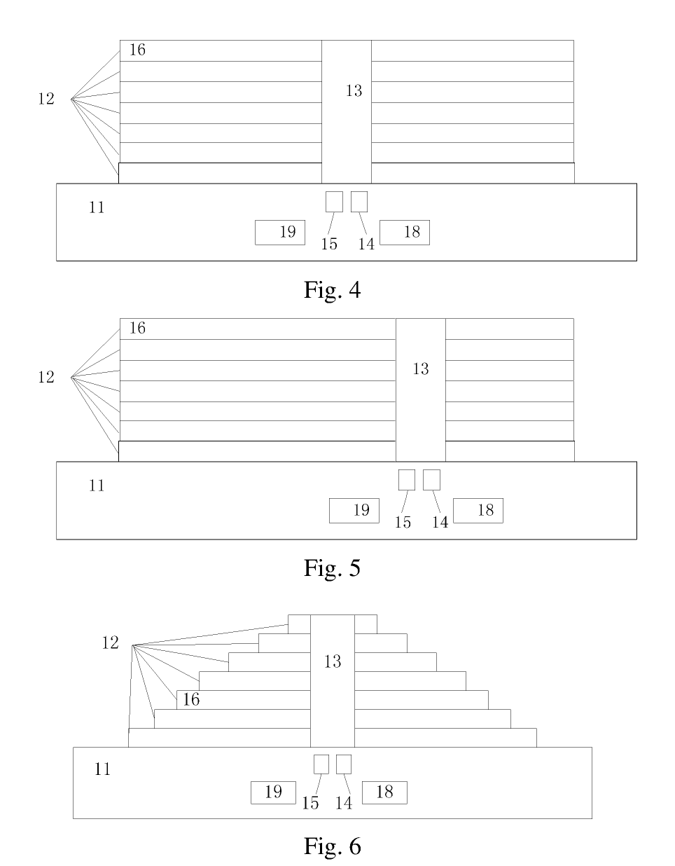

Referring to FIG. 4, a longitudinal cross section view of a photoelectric pulse wave sensor according to the fourth embodiment of the present disclosure is illustrated in FIG. 4. For brief, description of the same or similar technical features as those in any one of the above embodiments is incorporated herein and will not repeatedly described.

The photoelectric pulse wave sensor includes: a substrate 11, stacking layers 12, a transmission post 13, a measurement light source 14 and a photoelectric detector (PD) 15.

In the embodiment, the stacking layers 12 are disposed on the substrate 11 and are formed by stacking N layers of discs 16. Each of the discs 16 has a central hole. The central holes of the N layers of discs 16 are in positional correspondence with one another and form a through hole in the stacking layers that is perpendicular to the substrate, where 3≦N≦10. The measurement light source 14 and the photoelectric detector 15 are provided side-by-side on a face of the substrate that is right below the through hole, and a shape and a size of the transmission post 13 correspond to those of the through hole, and the transmission post 13 is fixed in the through hole.

As an example, the disc 16 is a rectangular disc, a circular disc, or an elliptical disc. As an example, the N is 7. The disc 17 is made of opacity material, such as acrylic.

As an example, the central hole of the disc may be in a shape of rectangular, circular or elliptical. Correspondingly, the transmission post 13 may be a cuboid, a circular cylinder or an elliptical pillar.

As an example, the N layers of disc may have the same thickness or may have different thicknesses from one another. The thickness of the disc may be in the range from 0.1 mm to 0.3 mm. When they have the same thickness, the thickness may be 0.2 mm.

Similar to the above embodiments, in the photoelectric pulse wave sensor according to the further embodiment of the present disclosure, a light emitted from the measurement light source 14 is reflected by blood and the reflected light is received by the photoelectric detector 15. As the photoelectric pulse wave sensor has the stacking layers 12 and the stacking layers 12 contact skin when they are pressed against locations including wrist or finger tip, the photoelectric pulse wave sensor applies a force to vascular wall through the stacking layers 12 such that pressures inside and outside the vascular wall are equal to each other and thus the pressure on the vascular wall is counteracted by the force applied by the stacking layers 12. That is, the pressure on the vascular wall will not affect the photoelectric detector 15 and a noise caused by the pressure on the vascular wall is eliminated, thereby increasing measurement accuracy of the pulse wave.

Referring to FIG. 5, a longitudinal cross section view of the photoelectric pulse wave sensor according to a fifth embodiment of the present disclosure is illustrated in FIG. 5. For brief, description of the same or similar technical features as those in any one of the above embodiments is incorporated herein and will not repeatedly described.

The through hole in the stacking layers of the photoelectric pulse wave sensor is not at a central position of the stacking layers, but deviates to a lateral side of the stacking layers.

The photoelectric pulse wave sensor according to the fifth embodiment of the present disclosure may be also provided such that pressures inside and outside of the vascular wall are equal to each other and thus the pressure on the vascular wall is counteracted by the force applied by the stacking layers 12. That is, the pressure on the vascular wall will not affect the photoelectric detector 15, and noise caused by the pressure on the vascular wall is eliminated, thereby increasing measurement accuracy of the pulse wave.

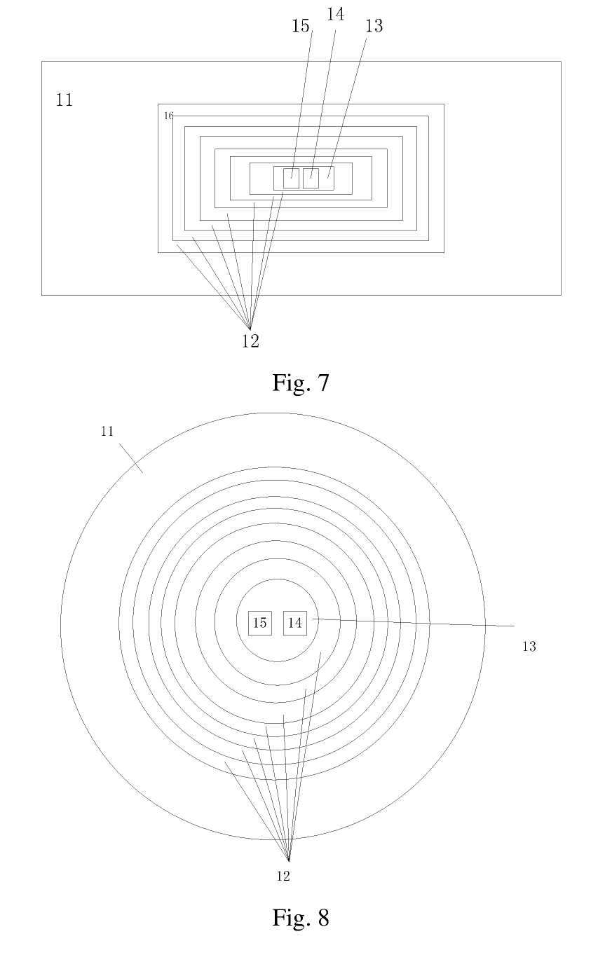

FIGS. 6 and 7 are respectively a longitudinal cross section view and a top view of a photoelectric pulse wave sensor according to the sixth embodiment of the present disclosure. For brief, description of the same or similar technical features as those in any one of the above embodiments is incorporated herein and will not repeatedly described.

Referring to FIGS. 6 and 7, sizes of cross sections of the N layers of discs decrease gradually layer by layer in a direction away from the substrate.

As an example, the sizes of the cross sections of the N layers of discs decrease gradually layer by layer in a way that a side length of an upper one of any two adjacent layers of discs is less than that of a lower one by 0.5 mm or more.

As an example, the N is 7 and the discs are rectangular discs, sizes of cross sections of the 7 layers of discs are respectively, from bottom to top, 20 mm×8 mm, 17 mm×7.5 mm, 14 mm×7 mm, 12 mm×6 mm, 10 mm×5 mm, 8 mm×4 mm, 6 mm×3 mm.

The photoelectric pulse wave sensor according to the sixth embodiment of the present disclosure may eliminate noise caused by the pressure on the vascular wall so as to increase measurement accuracy of the pulse wave, and further, make the external force sustained by the vascular wall near the position where measurement is performed by the sensor become more balance, and thus the pressure on the vascular wall may be counteracted more accurately by the external force imposed by the stacking layers 12, thereby further increasing measurement accuracy of the pulse wave.

Referring to FIG. 8, a top view of the photoelectric pulse wave sensor according to a seventh embodiment of the present disclosure is illustrated in FIG. 8. For brief, description of the same or similar technical features as those in any one of the above embodiments is incorporated herein and will not repeatedly described.

The stacking layers 12 of the photoelectric pulse wave sensor are formed by stacking N layers of discs. Similar to the above embodiments, the photoelectric pulse wave sensor according to the seventh embodiment of the present disclosure may eliminate noise caused by the pressure on the vascular wall so as to increase measurement accuracy of the pulse wave, and further, make the external force sustained by the vascular wall near the position where measurement is performed by the sensor become more balance, and thus the pressure on the vascular wall may be counteracted more accurately by the external force imposed by the stacking layers 12, thereby further increasing measurement accuracy of the pulse wave.

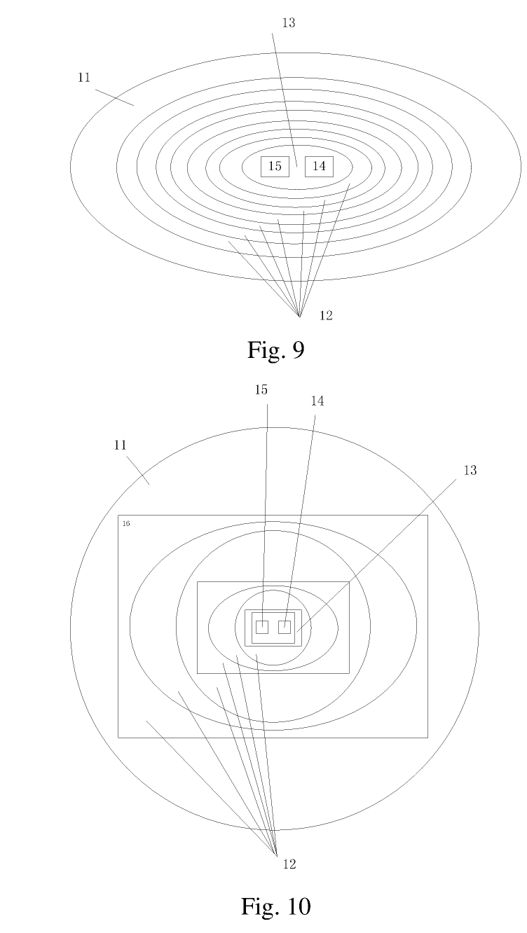

Referring to FIG. 9, a top view of the photoelectric pulse wave sensor according to an eighth embodiment of the present disclosure is illustrated in FIG. 9. For brief, description of the same or similar technical features as those in any one of the above embodiments is incorporated herein and will not repeatedly described.

The stacking layers 12 of the photoelectric pulse wave sensor are formed by stacking N layers of elliptical discs. Similar to the above embodiments, the photoelectric pulse wave sensor according to the eighth embodiment eliminates noise caused by the pressure on the vascular wall so as to increase measurement accuracy of the pulse wave, and further, make the external force sustained by the vascular wall near the position where measurement is performed by the sensor become more balance, and thus the pressure on the vascular wall may be counteracted more accurately by the external force imposed by the stacking layers 12, thereby further increasing measurement accuracy of the pulse wave.

Referring to FIG. 10, a top view of the photoelectric pulse wave sensor according to a ninth embodiment of the present disclosure is illustrated in FIG. 10. For brief, description of the same or similar technical features as those in any one of the above embodiments is incorporated herein and will not repeatedly described.

N layers of discs of the photoelectric pulse wave sensor are in shape of at least two of rectangular, circular and elliptical.

For example, in FIG. 10, the discs are respectively, from bottom to top, a rectangular disc, an elliptical disc, a circular disc, a rectangular disc, an elliptical disc, a circular disc and a rectangular disc. Similar to the above embodiments, the photoelectric pulse wave sensor according to the ninth embodiment eliminates noise caused by the pressure on the vascular wall so as to increases measurement accuracy of the pulse wave, and further, make the external force sustained by the vascular wall near the position where measurement is performed by the sensor become more balance, and thus the pressure on the vascular wall may be counteracted more accurately by the external force imposed by the stacking layers 12, thereby further increasing measurement accuracy of the pulse wave.

Hereto, the embodiments have been described in detailed in conjunction with the drawings. Based on the above description, those skilled in the art can obtain explicit understanding on the photoelectric pulse wave sensor of the present disclosure.

It can be learned from the above, the photoelectric pulse wave sensor of the present disclosure has advantages as followings:

the photoelectric pulse wave sensor includes the protrusion structure, the protrusion structure may be the pillar-shaped integral structure or the pillar-shaped stacking layers, and is configured such that pressures inside and outside vascular wall are equal to each other and thus the pressure on the vascular wall does not bring influence on the photoelectric detector, thereby eliminating noise caused by the pressure on the vascular wall and increasing measurement accuracy of the pulse wave;

the substantially tapered integral structure or stacking layers having multiple steps applies a maximum external force at a position of the vascular wall right facing the photoelectric detector and the external force on the vascular wall becomes smaller at position departing from the position right facing the photoelectric detector such that the external force sustained by the vascular wall near the position where measurement is performed by the sensor becomes more balance, and the pressure on the vascular wall may be more accurately counteracted by the external force applied by the stacking layers, thereby further increasing measure accuracy of the pulse wave.

Further, embodiments of the present disclosure provide a detection apparatus including the above described photoelectric pulse wave sensor. The detection apparatus may be a medical detection apparatus that is integrated with a plurality of functions (including a function of detecting pulse wave). The detection apparatus may also be various wearable products or mobile apparatuses which have a function of health inspection or monitor, and it is not limited by the embodiments of the present disclosure.

It is noted that all embodiments that are not illustrated or described in the drawings or the description are known by those skilled in the art and are not described in detailed herein. In addition, definitions of the above components are not limited to these specific structures, shapes or manners introduced in the above embodiments and may be modified or replaced simply by those skilled in the art. For example:

the disc or the step may be in other shape;

the directional or orientational terms introduced in the embodiments such as “upper”, “lower”, “front”, “back”, “right” and “left” are only defined in terms of the accompanying drawings, instead of limiting the protective scope of the present invention;

these above embodiments may be combined with each other or may be combined with other embodiment(s) depending on design and reliability, i.e., technical features of different embodiments may be freely combined to form further more embodiments.

The objects, technical solutions and advantages of the present disclosure are further described in detailed in the above specific embodiments. It is understood that the above is only specific embodiments of the present invention, instead of limiting scope of the present invention. Any modification(s), replacement(s) and improvement(s), which are within principles and spirit of the present invention, on the above embodiments shall be included in the protective scope of the present invention.

1. A photoelectric pulse wave sensor comprising: a substrate, a protrusion structure, a transmission post, a measurement light source and a photoelectric detector, wherein,
the protrusion structure is protrudedly disposed on the substrate and has a through hole therein that is perpendicular to the substrate; and
the measurement light source and the photoelectric detector are provided side-by-side on a face of the substrate that is right below the through hole, a shape and a size of the transmission post are matched to a shape and a size of the through hole and the transmission post is fixed in the through hole.

2. The photoelectric pulse wave sensor according to claim 1, wherein, the protrusion structure includes stacking layers that are composed of N layers of discs each having a central hole, the central holes of the N layers of discs being in positional correspondence with one another and forming the through hole that is perpendicular to the substrate, where N≧2.

3. The photoelectric pulse wave sensor according to claim 2, wherein, 3≦N≦10.

4. The photoelectric pulse wave sensor according to claim 2, wherein, sizes of cross sections, perpendicular to a protrusion direction, of the N layers of discs decrease gradually layer by layer in a direction away from the substrate, such that the N layers of discs form a substantially tapered protrusion structure.

5. The photoelectric pulse wave sensor according to claim 4, wherein, the disc is a rectangular disc, a circular disc or an elliptical disc.

6. The photoelectric pulse wave sensor according to claim 2, wherein, the N layers of discs have a same shape, and cross sections, perpendicular to the protrusion direction, of the N layers of discs have a same size.

7. The photoelectric pulse wave sensor according to claim 6, wherein, the disc is a rectangular disc, a circular disc or an elliptical disc, to form a pillar-shaped protrusion structure as a whole.

8. The photoelectric pulse wave sensor according to claim 1, wherein, the protrusion structure is a pillar-shaped integral structure or a substantially tapered integral structure having multiple steps.

9. The photoelectric pulse wave sensor according to claim 8, wherein, the protrusion structure is a substantially tapered integral structure and comprises N step structures; and sizes of cross sections, perpendicular to a protrusion direction, of the N step structures decrease gradually layer by layer in a direction away from the substrate.

10. The photoelectric pulse wave sensor according to claim 9, wherein, a cross section, perpendicular to the protrusion direction, of each of the N step structures has a shape of rectangle, circle or ellipse.

11. The photoelectric pulse wave sensor according to claim 1, wherein, the through hole in the protrusion structure is located in a central position of the protrusion structure or is deviated to a side of the protrusion structure.

12. The photoelectric pulse wave sensor according to claim 1, wherein, a cross section of the through hole in the protrusion structure is in a shape of rectangle, circle or ellipse, and the transmission post is correspondingly in a shape of a cuboid, a circular cylinder or an elliptical pillar.

13. The photoelectric pulse wave sensor according to claim 1, further comprising:
a constant-current source control circuit located in the substrate, connected to the measurement light source and configured such that an electrical current flowing through the measurement light source is kept to be constant and thus the measurement light source emits a light with stable intensity.

14. The photoelectric pulse wave sensor according to claim 1, further comprising:
a signal modulation circuit located in the substrate, connected to the photoelectric detector and configured to filter out a direct-current component of an output signal of the photoelectric detector.

15. The photoelectric pulse wave sensor according to claim 1, wherein the protrusion structure has a greatest thickness at a position right facing the photoelectric detector, and the thickness of the protrusion structure decreases gradually as departing from the position right facing the photoelectric detector, thereby the protrusion structure presenting a substantially tapered shape.

16. A detection apparatus comprising the photoelectric pulse wave sensor according to claim 1.

17. A detection apparatus comprising the photoelectric pulse wave sensor according to claim 2.

18. A detection apparatus comprising the photoelectric pulse wave sensor according to claim 8.

19. A detection apparatus comprising the photoelectric pulse wave sensor according to claim 11.

20. A detection apparatus comprising the photoelectric pulse wave sensor according to claim 12.
